(19) United States
(12) Patent Application Publication
(10) Pub. No.: US 2020/0215342 A1
(43) Pub. Date: Jul. 9, 2020

(54) SECURING IMPLANTED TISSUE STIMULATORS TO SURROUNDING TISSUES

(71) Applicant: Stimwave Technologies Incorporated, Pompano Beach, FL (US)

(72) Inventors: Laura Tyler Perryman, Pompano Beach, FL (US); Graham Patrick Greene, Miami Beach, FL (US); Chad David Andresen, Miami Beach, FL (US)

(21) Appl. No.: 16/691,761

(22) Filed: Nov. 22, 2019

Related U.S. Application Data
(60) Provisional application No. 62/790,128, filed on Jan. 9, 2019.

(51) Int. Cl. A61N 1/375

(52) U.S. Cl. CPC A61N 1/37518; A61N 1/3756

(57) ABSTRACT

A device for securing a tissue stimulator to a tissue within a body includes a first housing and a second housing. The first housing defines a first receptacle configured to grasp the tissue stimulator and a protrusion extending away from the receptacle. The second housing defines a second receptacle configured to grasp the tissue stimulator and an opening configured to receive the protrusion to secure the first and second housings together such that the first and second receptacles are aligned to form a channel that surrounds the tissue stimulator and fixes a position of the tissue stimulator relative to the first and second housings. The device further includes an attachment feature by which either or both of the first and second housings can be secured to the tissue with the tissue stimulator carried therein.

### CROSS-REFERENCE TO RELATED APPLICATION

This application claims the benefit of U.S. Provisional Application No. 62/790,128, filed Jan. 9, 2019, and titled “Securing Implanted Tissue Stimulators to Surrounding Tissues,” which is incorporated by reference.

### TECHNICAL FIELD

This disclosure relates to devices for securing implanted tissue stimulators to surrounding tissues within a body, such as within a subcutaneous space.

### BACKGROUND

Modulation of tissue within the body by electrical stimulation has become an important type of therapy for treating chronic, disabling conditions, such as chronic pain, problems of movement initiation and control, involuntary movements, dystonia, urinary and fecal incontinence, sexual difficulties, vascular insufficiency, and heart arrhythmia. For example, an external antenna can be used to send electrical energy to electrodes on an implanted tissue stimulator that can pass pulsatile electrical currents of controllable frequency, pulse width, and amplitudes to a tissue. In order to deliver a desired therapy to the tissue, the tissue stimulator should be optimally positioned with respect to the tissue in a secure manner.

### SUMMARY

In general, this disclosure relates to devices for securing implanted tissue stimulators to surrounding tissues within a body, such as within a subcutaneous space. Such tissue stimulators are designed to deliver electrical therapy to the surrounding tissues.

In one aspect, a device for securing a tissue stimulator to a tissue within a body includes a first housing and a second housing. The first housing defines a first receptacle configured to grasp the tissue stimulator and a protrusion extending away from the receptacle. The second housing defines a second receptacle configured to grasp the tissue stimulator and an opening configured to receive the protrusion to secure the first and second housings together such that the first and second receptacles are aligned to form a channel that surrounds the tissue stimulator and fixes a position of the tissue stimulator relative to the first and second housings. The device further includes an attachment feature by which either or both of the first and second housings can be secured to the tissue with the tissue stimulator carried therein..

Embodiments may provide one or more of the following features.

In some embodiments, the tissue stimulator is a first tissue stimulator, the channel is a first channel, and the first housing further defines a third receptacle configured to grasp a second tissue stimulator and the second housing further defines a fourth receptacle configured to grasp the second tissue stimulator such the third and fourth receptacles are aligned to form a second channel that surrounds the second tissue stimulator to fix the position of the first tissue stimulator relative to the second tissue stimulator.

In some embodiments, the first and second channels are spaced apart from each other.

In some embodiments, the first and second channels are spaced apart by a distance of about 2 mm to about 15 mm.

In some embodiments, the first and second channels have a length of about 2 mm to about 30 mm.

In some embodiments, the attachment feature includes a through hole by which the device can be sutured to the tissue.

In some embodiments, the attachment feature includes a textured surface into which the tissue can grow to secure the device to the tissue.

In some embodiments, the attachment feature is defined by one or both of the first and second housings.

In some embodiments, the first and second housings include one or more biocompatible polymer materials.

In some embodiments, the first and second housings have a durometer of 40 Shore A to 90 Shore D.

In some embodiments, the device further includes multiple gripping elements disposed along the first and second receptacles for grasping the tissue stimulator.

In some embodiments, the multiple gripping elements include teeth.

In some embodiments, the multiple gripping elements have a convex profile.

In some embodiments, the protrusion is configured to clamp the first housing to the second housing.

In some embodiments, the protrusion includes a lip configured to remain exterior to the opening in the second housing when the first and second housings are attached to each other.

In another aspect, a method of using a device to secure a tissue stimulator to a tissue within a body includes grasping the tissue stimulator within a first receptacle of a first housing of the device, passing a protrusion of the first housing through an opening of a second housing of the device to attach the second housing to the first housing such that a second receptacle of the second housing grasps the tissue stimulator and such that the first and second receptacles align to form a channel that surrounds the tissue stimulator and fixes a position of the tissue stimulator relative to the first and second housings, and securing either or both of the first and second housings, with the tissue stimulator carried therein, to the tissue at an attachment feature of the device.

Embodiments may provide one or more of the following features.

In some embodiments, the tissue stimulator is a first tissue stimulator and the channel is a first channel, method further including grasping a second tissue stimulator within a third receptacle of the first housing of the device, and grasping the second tissue stimulator within a fourth receptacle of the second housing of the device such that the third and fourth receptacles align to form a second channel that surrounds the second tissue stimulator and fixes the position of the first tissue stimulator relative to the second tissue stimulator.

In some embodiments, the method further includes positioning the first and second tissue stimulators about 2 mm to about 15 mm from each other.

In some embodiments, the method further includes suturing one or both of the first and second housings at a through opening of the device.

In some embodiments, the attachment feature includes a textured surface into which the tissue can grow to secure the device to the tissue.

### DESCRIPTION OF DRAWINGS

FIG. 1 is an exploded perspective view of an implantable device designed to position tissue stimulators relative to each other and relative to a nearby tissue.

FIG. 2 is a perspective view of the implantable device of FIG. 1 in an assembled state.

FIG. 3 is a cross-sectional view of protrusions that extend from receptacles of the implantable device of FIG. 1.

FIG. 4 is a top view of the tissue stimulators positioned in alignment with each other within the implantable device of FIG. 1.

FIG. 5 is a top view of the tissue stimulators positioned in an offset configuration within the implantable device of FIG. 1.

FIG. 6 is an exploded perspective view of an implantable device designed to position tissue stimulators relative to each other and relative to a nearby tissue.

FIG. 7 is a perspective view of the implantable device of FIG. 6 in an assembled state.

FIG. 8 is a cross-sectional view of a surface profile of the implantable device of FIG. 6.

FIG. 9 is a flowchart of a method of using the implantable device of FIG. 1 or the implantable device of FIG. 6 to secure a tissue stimulator to a tissue within a body.

FIG. 10 is a system block diagram of a neural stimulation system including the tissue stimulators of FIGS. 1 and 6.

FIG. 11 is a detailed block diagram of the neural stimulation system of FIG. 10.

### DETAILED DESCRIPTION

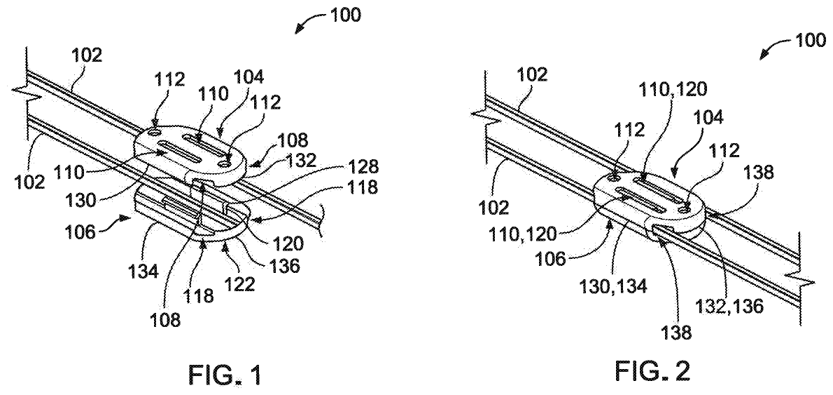

FIGS. 1 and 2 illustrate an implantable device 100 designed to fix positions of two tissue stimulators 102 relative to each other and to fix the positions of the tissue stimulators 102 relative to a nearby (e.g., surrounding or adjacent) tissue while the tissue stimulators 102 are implanted within the body. The implantable device 100 includes an upper housing 104 and a lower housing 106 that are formed to mate with each other to grasp the implantable tissue stimulators 102, thereby fixing their relative positions.

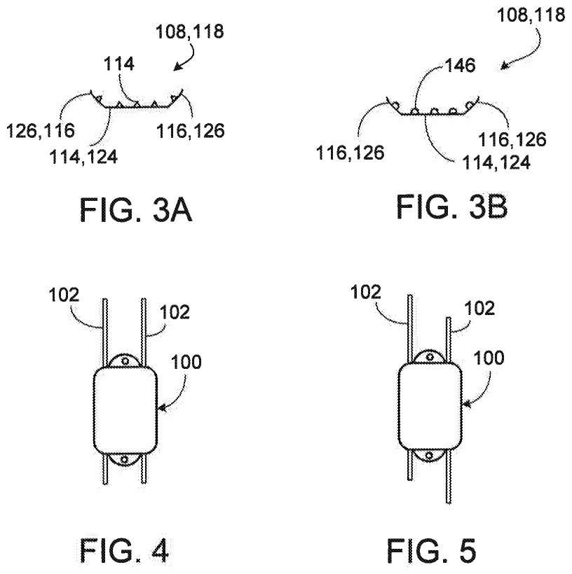

The upper housing 104 defines two receptacles 108 sized to receive the tissue stimulators 102, two recessed slits 110 (e.g., elongate openings) formed to mate with the lower housing 106, and two through openings 112 by which the implantable device 100 can be attached (e.g., sutured or otherwise anchored) to the surrounding tissue. The receptacles 108 extend along straight lateral sides 130 of the upper housing 104 and have a flat base surface 114 and curved lateral surfaces 116 (shown in FIG. 3). The through openings 112 are positioned along curved ends 132 of the upper housing 104.

The lower housing 106 also defines two receptacles 218 that are sized to receive the tissue stimulators 102, two protrusions 120 formed to mate with the slits 110 of the upper housing 104, and two through openings 122 by which the implantable device 100 can be attached to the surrounding tissue. The receptacles 218 extend along straight lateral sides 134 of the lower housing 106 and have a flat base surface 124 and curved lateral surfaces 126. The through openings 122 are positioned along curved ends 136 of the lower housing 106.

The upper housing 104 can be attached to the lower housing 106 to secure the tissue stimulators 102, positioned snuggly within the receptacles 218. When the upper housing 104 is clamped (e.g., pressed down) against the lower housing 106, the upper and lower receptacles 108, 118 together define channels 138 that surround and grasp the tissue stimulators 102 via friction fit to prevent sliding of the tissue stimulators 102 out of or off of the implantable device 100. Additionally, the protrusions 120 pass through the slits 110, and the through openings 112 are respectively aligned with the through openings 122. The protrusions 120 respectively define flanges 128 (e.g., lips) that retain the protrusions 120 external to the slits 110 to maintain attachment between the upper and lower housings 104, 106.

Referring to FIG. 3, the implantable device 100 can further include protrusions 144, 146 (e.g., gripping elements) that extend along one or more of the receptacles 108, 118 to grasp the tissue stimulators 102 therein. Example protrusions 144, 146 include teeth (a) and convex projections (b), among others.

The upper and lower housings 104, 106 are typically made of one or more biocompatible materials, such as polyurethane (e.g., aromatic polyether-based thermoplastic polyurethanes), silicone, and epoxy. For example, the materials from which the upper and lower housings 104, 106 typically have a durometer of 40 Shore A to 90 Shore D. The upper and lower housings 104, 106 are typically manufactured via injection molding or computer numerical control techniques. The implantable device 100 typically has a length of about 3 mm to about 30 mm, a width of about 5 mm to about 20 mm, and a thickness of about 1.0 mm to about 3.0 mm. The lateral sides 130, 134 typically have a length of about 2 mm to about 28 mm. The channels 138 typically have a length (e.g., terminating at the curved ends 132, 136) of about 2 mm to about 28 mm. The channels 138 typically have a width about 0.5 mm to about 3.0 mm and a thickness of about 0.5 mm to about 3.0 mm. The channels 138 (e.g., and the tissue stimulators 102 secured therein) are typically spaced apart by about 2.0 mm to about 15 mm (e.g., about 10 mm). Such spacing between the tissue stimulators 102 is optimized based on the transmission frequency and improves reception to both tissue stimulators 102. The through openings 112, 122 typically have a diameter of about 0.5 mm to about 3.0 mm.

In order to deploy the implantable device 100 to the tissue stimulators 102 within the body, an incision 140 is made adjacent an implantation site 142 (e.g., a subcutaneous space) of the tissue stimulators 102 (shown in FIGS. 4 and 5). The tissue stimulators 102 are snuggly positioned at desired locations along their lengths within the receptacles 218 of the lower housing 106 and then optionally slid within the receptacles 218 to make minor adjustments to their positioning. The upper housing 104 is subsequently clamped onto the lower housing 106 to secure the tissue stimulators 102 in place within the channels 138, and the implantable device 100, assembled with the tissue stimulators 102, is itself positioned at or moved to a desired location at the implantation site 142. Sutures are then passed through the through openings 112, 122 and the surrounding tissue and tied to attach (e.g., anchor) the implantable device 100 to the surrounding tissue at a desired a location. Such attachment of the implantable device 100 to the surrounding tissue prevents or reduces bunching or coiling of the tissue stimulators 102 within a subcutaneous space or fat tissue. In some examples, the tissue stimulators 102 are positioned within the channels 138 at the same position such that respective ends 102 of the tissue stimulators 102 are aligned, as shown in FIG. 4. In other examples, the tissue stimulators 102 are positioned within the channels 138 at different positions such that respective ends 102 of the tissue stimulators 102 are offset from each other, as shown in FIG. 5. The tissue stimulators 102 may have the same length or different lengths. In some implementations, the housings 104, 106 be disengaged to reposition the tissue stimulators 102 if desired.

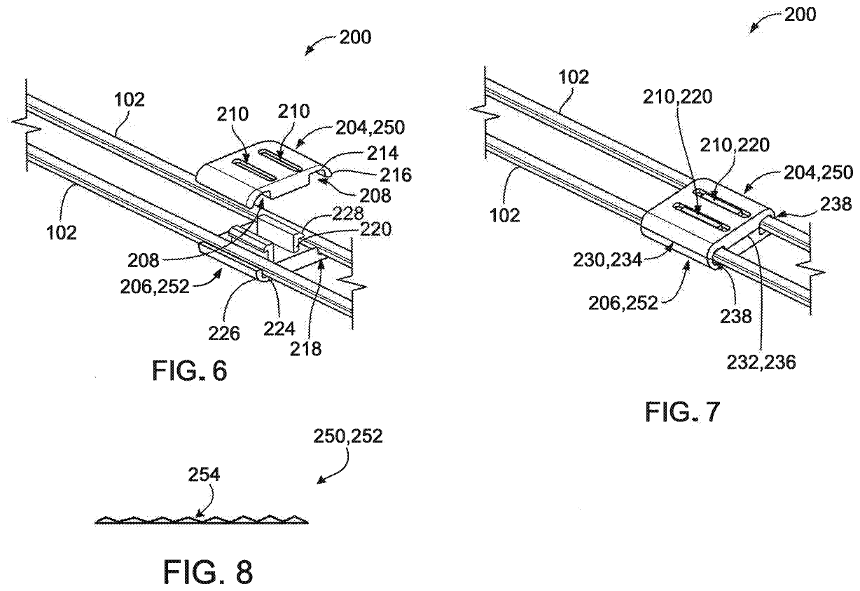

While the implantable device 100 has been described and illustrated as including through openings 112, 122 for attachment (e.g., via suturing) to a surrounding tissue, in some embodiments, a positioning device does not include the through openings (e.g., and therefore lacks the associated tension points) and instead includes a different anchoring feature for attachment to a surrounding tissue. For example, FIGS. 6 and 7 illustrate an implantable device 200 that includes textured exterior surfaces for attachment to a surrounding tissue. The implantable device 200 includes an upper housing 204 and a lower housing 206 that are formed to mate with each other to grasp the implantable tissue stimulators 102, thereby fixing their relative positions.

The upper housing 204 defines two receptacles 208 sized to receive the tissue stimulators 102, two recessed slits 210 (e.g., elongate openings) formed to mate with the lower housing 206, and a textured surface 250 by which the implantable device 200 can be attached or secured to the surrounding tissue. The receptacles 208 extend along lateral sides 230 of the upper housing 204 between straight ends 232, as the upper housing 204 lacks a curved end with a through opening. The receptacles 208 have a flat base surface 214 and curved lateral surfaces 216.

The lower housing 206 also defines two channels 218 that are sized to receive the tissue stimulators 102, two protrusions 220 formed to mate with the slits 210 of the upper housing 204, and a textured surface 252 by which the implantable device 200 can be attached or secured to the surrounding tissue. The channels 218 extend along lateral sides 234 of the lower housing 206 between straight ends 236. The channels 218 have a flat base surface 224 and curved lateral surfaces 226.

The upper housing 204 can be attached to the lower housing 206 to secure the tissue stimulators 102, positioned snuggly within the channels 218. When the upper housing 204 is clamped (e.g., pressed down against) the lower housing 206, the upper and lower receptacles 208, 218 together define channels 238 that surround and grasp the tissue stimulators 102 via friction fit to prevent sliding of the tissue stimulators 102 out of or off of the implantable device 200. Additionally, the protrusions 220 pass through the slits 210. The protrusions 220 respectively define flanges 228 (e.g., lips) that retain the protrusions 220 external to the slits 210 to maintain attachment between the upper and lower housings 204, 206. The implantable device 200 can further include the protrusions 144, 146 (shown in FIG. 3) along one or more of the receptacles 208, 218 to grasp the tissue stimulators 102 therein.

Referring to FIG. 8, the textured surfaces 250, 252 may be formed as “bumpy” profiles with recessions 254 into which a surrounding tissue (e.g., scar tissue) can grow to stably secure the implantable device 200 to the tissue. For example, in some embodiments, the bumpy profiles may include spherical or triangular features for promoting tissue ingrowth.

The upper and lower housings 204, 206 are typically made of the same materials from which the upper and lower housings 104, 106 are made. For example, the materials from which the upper and lower housings 204, 206 typically have a durometer of 40 Shore A to 90 Shore D. The upper and lower housings 204, 206 are typically manufactured via injection molding or computer numerical control techniques. In some embodiments, the textured surfaces 250, 252 may be provided as separate layers that coat the upper and lower housings 204, 206. In such embodiments, the layers may have the same material composition as the upper and lower housings 204, 206 or have different material compositions. For example, in some embodiments, the layers may include one or more materials that are bumpy and/or providing gripping to promote tissue ingrowth, such as silicone. The implantable device 200 typically has a length of about 3 mm to about 25 mm, a width of about 5 mm to about 20 mm, and a thickness of about 1.0 mm to about 3.0 mm. The channels 238 typically have a width about 0.5 mm to about 3.0 mm and a thickness of about 0.5 mm to about 3.0 mm. The channels 238 (e.g., and the tissue stimulators 102 secured therein) are typically spaced apart by about 2.0 mm to about 15 mm (e.g., about 10 mm).

In order to deploy the implantable device 200 to the tissue stimulators 102 within the body, the tissue stimulators 102 are snuggly clamped between the upper and lower housings 204, 206, as described above with respect to the implantable device 100, and the implantable device 200, assembled with the tissue stimulators 102, is itself positioned at or moved to a desired location at the implantation site 142. Surrounding tissue may then grow into and be retained in recessed regions of the textured surfaces 250, 252 to secure the implantable device 200 to the tissue.

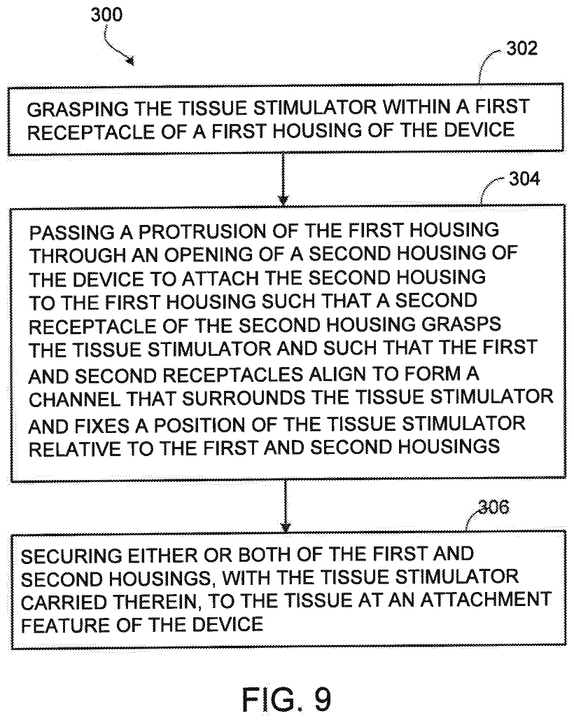

FIG. 9 provides a flowchart that illustrates a method 300 of using an implantable device (e.g., the implantable device 100, 200) to secure a tissue stimulator (e.g., the tissue stimulator 102) to a tissue within a body. In some examples, the method includes grasping the tissue stimulator within a first receptacle (e.g., the receptacle 118, 218) of a first housing (e.g., the lower housing 106, 206) of the device (302). In some examples, the method further includes passing a protrusion (e.g., the protrusion 120, 220) of the first housing through an opening (e.g., the slit 110, 210) of a second housing of the device to attach the second housing to the first housing such that a second receptacle (e.g., the receptacle 108, 208) of the second housing grasps the tissue stimulator and such that the first and second receptacles align to form a channel (e.g., the channel 138, 238) that surrounds the tissue stimulator and fixes a position of the tissue stimulator relative to the first and second housings (304). In some examples, the method further includes securing either or both of the first and second housings, with the tissue stimulator carried therein, to the tissue at an attachment feature (e.g., the through openings 112, 122 or the textured surfaces 250, 252) of the device (306).

While the implantable devices 100, 200 have been described and illustrated as including certain dimensions, sizes, shapes, materials, arrangements, and configurations, in some embodiments, positioning devices that are similar in structure and function to either of the implantable devices 100, 200 may include different dimensions, sizes, shapes, materials, arrangements, or configurations.

While the implantable devices 100, 200 have been described and illustrated as including two channels 138, 238 for positioning and attaching two tissue stimulators 102, in some embodiments, a positioning device that is similar in structure and function to either of the implantable devices 100, 200 may include only a single slot for positioning a single tissue stimulator 102 and attaching the tissue stimulator 102 to a surrounding tissue. In alternative embodiments, a positioning device that is similar in structure and function to either of the implantable devices 100, 200 may include more than two slots for respectively positioning more than two tissue stimulators 102 and attaching the tissue stimulators 102 to a surrounding tissue. In some embodiments, a positioning device that is similar in structure and function to either of the implantable devices 100, 200 may include only a single protrusion 120, 220 and a corresponding slit 110, 210 or may include more than two protrusions 120, 220 and corresponding slits 110, 210.

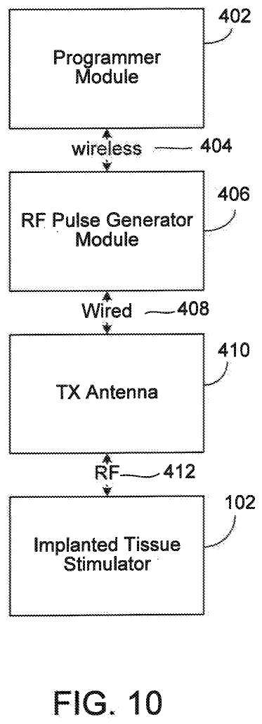

In some embodiments, a tissue stimulator 102 (e.g., a wireless tissue stimulator) may be provided as part of a tissue stimulation system, such as a neural stimulation system 400. Referring to FIG. 10, the neural stimulation system 400 is designed to send electrical pulses to a nearby (e.g., adjacent or surrounding) target nerve tissue to stimulate the target nerve tissue by using remote radio frequency (RF) energy without cables and without inductive coupling to power the tissue stimulator 102. Accordingly, the tissue stimulator 102 is provided as a passive tissue stimulator in the neural stimulation system 400. In some examples, the target nerve tissue is in the spinal column and may include one or more of the spinothalamic tracts, the dorsal horn, the dorsal root ganglia, the dorsal roots, the dorsal column fibers, and the peripheral nerves bundles leaving the dorsal column or the brainstem. In some examples, the target nerve tissue may include one or more of cranial nerves, abdominal nerves, thoracic nerves, trigeminal ganglia nerves, nerve bundles of the cerebral cortex, deep brain, sensory nerves, and motor nerves.

The neural stimulation system 400 further includes a programmer module 402, an RF pulse generator module 406 (e.g., a controller module), and a transmit (TX) antenna 410. In some embodiments, the programmer module 502 is a computing device (e.g., a smart phone, another mobile computing device, or a stationary computing device) running a software application that supports a wireless connection 404 (e.g., via Bluetooth.R™). The software application can enable the user to view a system status and system diagnostics, change various parameters, increase and decrease a desired stimulus amplitude of the electrical pulses, and adjust a feedback sensitivity of the RF pulse generator module 406, among other functions.

The RF pulse generator module 406 includes stimulation circuitry, a battery to power generator electronics, and communication electronics that support the wireless connection 404. In some embodiments, the RF pulse generator module 406 is designed to be worn external to the body, and the TX antenna 410 (e.g., located external to the body) is connected to the RF pulse generator module 406 by a wired connection 508. Accordingly, the RF pulse generator module 406 and the TX antenna 410 may be incorporated into a wearable accessory (e.g., a belt or a harness design) or a clothing article such that electric radiative coupling can occur through the skin and underlying tissue to transfer power and/or control parameters to the tissue stimulator 102.

The TX antenna 410 can be coupled directly to tissues within the body to create an electric field that powers the implanted tissue stimulator 102. The TX antenna 410 communicates with the tissue stimulator 102 through an RF interface. For instance, the TX antenna 410 radiates an RF transmission signal that is modulated and encoded by the RF pulse generator module 406. The tissue stimulator 102 includes one or more antennas (e.g., dipole antennas) that can receive and transmit through an RF interface 412. In particular, the coupling mechanism between the TX antenna 410 and the one or more antennas on the tissue stimulator 102 is electrical radiative coupling and not inductive coupling. In other words, the coupling is through an electric field rather than through a magnetic field. Through this electrical radiative coupling, the TX antenna 410 can provide an input signal to the tissue stimulator 102.

In addition to the one or more antennas, the tissue stimulator 102 further includes internal receiver circuit components that can capture the energy carried by the input signal sent from the TX antenna 104 and demodulate the input signal to convert the energy to an electrical waveform. The receiver circuit components can further modify the waveform to create electrical pulses suitable for stimulating the target neural tissue. The tissue stimulator 102 further includes electrodes that can deliver the electrical pulses to the target neural tissue. For example, the circuit components may include wave conditioning circuitry that rectifies the received RF signal (e.g., using a diode rectifier), transforms the RF energy to a low frequency signal suitable for the stimulation of neural tissue, and presents the resulting waveform to an array of the electrodes. In some implementations, the power level of the input signal directly determines an amplitude (e.g., a power, a current, and/or a voltage) of the electrical pulses applied to the target neural tissue by the electrodes. For example, the input signal may include information encoding stimulus waveforms to be applied at the electrodes.

In some implementations, the RF pulse generator module 406 can remotely control stimulus parameters of the electrical pulses applied to the target neural tissue by the electrodes and monitor feedback from the tissue stimulator 102 based on RF signals received from the tissue stimulator 102. For example, a feedback detection algorithm implemented by the RF pulse generator module 406 can monitor data sent wirelessly from the tissue stimulator 102, including information about the energy that the tissue stimulator 102 is receiving from the RF pulse generator 406 and information about the stimulus waveform being delivered to the electrodes. Accordingly, the circuit components internal to the tissue stimulator 102 may also include circuitry for communicating information back to the RF pulse generator module 406 to facilitate the feedback control mechanism. For example, the tissue stimulator 102 may send to the RF pulse generator module 406 a stimulus feedback signal that is indicative of parameters of the electrical pulses, and the RF pulse generator module 406 may employ the stimulus feedback signal to adjust parameters of the signal sent to the tissue stimulator 102.

In order to provide an effective therapy for a given medical condition, the neural stimulation system 400 can be tuned to provide the optimal amount of excitation or inhibition to the nerve fibers by electrical stimulation. A closed loop feedback control method can be used in which the output signals from the tissue stimulator 102 are monitored and used to determine the appropriate level of neural stimulation current for maintaining effective neuronal activation. Alternatively, in some cases, the patient can manually adjust the output signals in an open loop control method.

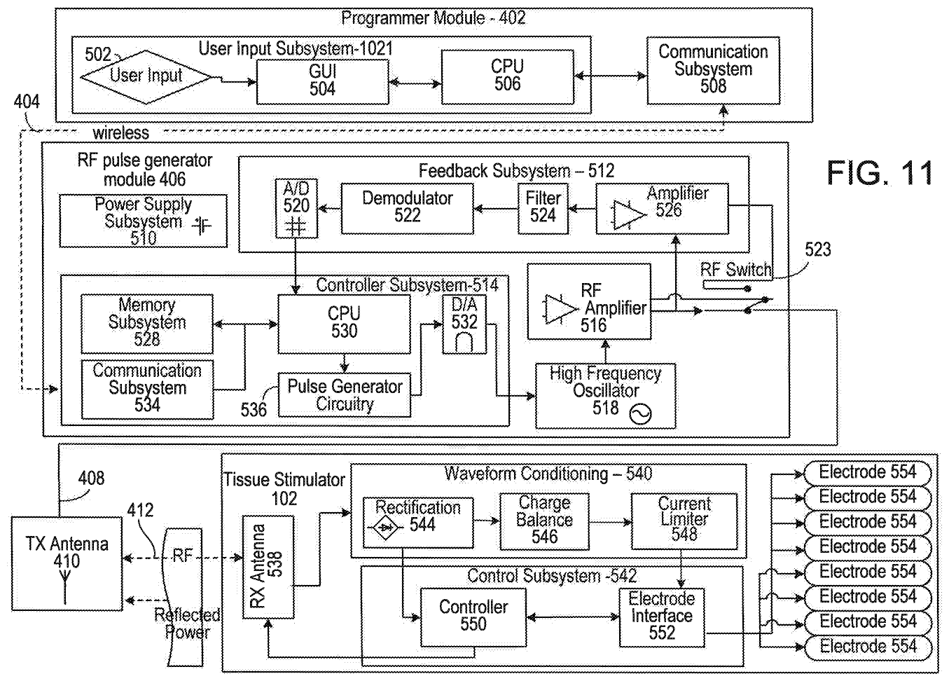

FIG. 11 depicts a detailed diagram of the neural stimulation system 400. The programmer module 402 may be used as a vehicle to handle touchscreen input on a graphical user interface (GUI) 204 and may include a central processing unit (CPU) 206 for processing and storing data. The programmer module 402 includes a user input system 521 and a communication subsystem 508. The user input system 521 can allow a user to input or adjust instruction sets in order to adjust various parameter settings (e.g., in some cases, in an open loop fashion). The communication subsystem 508 can transmit these instruction sets (e.g., and other information) via the wireless connection 404 (e.g., via a Bluetooth or Wi-Fi connection) to the RF pulse generator module 406. The communication subsystem 508 can also receive data from RF pulse generator module 406.

The programmer module 402 can be utilized by multiple types of users (e.g., patients and others), such that the programmer module 402 may serve as a patient's control unit or a clinician's programmer unit. The programmer module 402 can be used to send stimulation parameters to the RF pulse generator module 406. The stimulation parameters that can be controlled may include a pulse amplitude in a range of 0 mA to 20 mA, a pulse frequency in a range of 0 Hz to 2000 Hz, and a pulse width in a range of 0 ms to 2 ms. In this context, the term pulse refers to the phase of the waveform that directly produces stimulation of the tissue. Parameters of a charge-balancing phase (described below) of the waveform can similarly be controlled. The user can also optionally control an overall duration and a pattern of a treatment.

The tissue stimulator 102 or the RF pulse generator module 406 may be initially programmed to meet specific parameter settings for each individual patient during an initial implantation procedure. Because medical conditions or the body itself can change over time, the ability to readjust the parameter settings may be beneficial to ensure ongoing efficacy of the neural modulation therapy.

Signals sent by the RF pulse generator module 406 to the tissue stimulator 102 may include both power and parameter attributes related to the stimulus waveform, amplitude, pulse width, and frequency. The RF pulse generator module 406 can also function as a wireless receiving unit that receives feedback signals from the tissue stimulator 102. To that end, the RF pulse generator module 406 includes microelectronics or other circuitry to handle the generation of the signals transmitted to the tissue stimulator 102, as well as feedback signals received from tissue stimulator 102. For example, the RF pulse generator module 406 includes a controller subsystem 514, a high-frequency oscillator 518, an RF amplifier 516, an RF switch, and a feedback subsystem 512.

The controller subsystem 514 includes a CPU 530 to handle data processing, a memory subsystem 528 (e.g., a local memory), a communication subsystem 534 to communicate with the programmer module 402 (e.g., including receiving stimulation parameters from the programmer module 402), pulse generator circuitry 536, and digital/analog (D/A) converters 532.

The controller subsystem 514 may be used by the user to control the stimulation parameter settings (e.g., by controlling the parameters of the signal sent from RF pulse generator module 406 to tissue stimulator 102). These parameter settings can affect the power, current level, or shape of the electrical pulses that will be applied by the electrodes. The programming of the stimulation parameters can be performed using the programming module 402 as described above to set a repetition rate, pulse width, amplitude, and waveform that will be transmitted by RF energy to a receive (RX) antenna 538 (e.g., or multiple RX antennas 538) within the tissue stimulator 102. The RX antenna 538 may be a dipole antenna or another type of antenna. A clinician user may have the option of locking and/or hiding certain settings within a programmer interface to limit an ability of a patient user to view or adjust certain parameters since adjustment of certain parameters may require detailed medical knowledge of neurophysiology, neuroanatomy, protocols for neural modulation, and safety limits of electrical stimulation.

The controller subsystem 514 may store received parameter settings in the local memory subsystem 528 until the parameter settings are modified by new input data received from the programmer module 402. The CPU 506 may use the parameters stored in the local memory to control the pulse generator circuitry 536 to generate a stimulus waveform that is modulated by the high frequency oscillator 518 in a range of 300 MHz to 8 GHz. The resulting RF signal may then be amplified by an RF amplifier 526 and sent through an RF switch 523 to the TX antenna 410 to reach the RX antenna 538 through a depth of tissue.

In some implementations, the RF signal sent by the TX antenna 410 may simply be a power transmission signal used by tissue stimulator 102 to generate electric pulses. In other implementations, the RF signal sent by the TX antenna 410 may be a telemetry signal that provides instructions about various operations of the tissue stimulator 102. The telemetry signal may be sent by the modulation of the carrier signal through the skin. The telemetry signal is used to modulate the carrier signal (e.g., a high frequency signal) that is coupled to the antenna 238 and does not interfere with the input received on the same lead to power the tissue stimulator 102. In some embodiments, the telemetry signal and the powering signal are combined into one signal, where the RF telemetry signal is used to modulate the RF powering signal such that the tissue stimulator 102 is powered directly by the received telemetry signal. Separate subsystems in the tissue stimulator 102 harness the power contained in the signal and interpret the data content of the signal.

The RF switch 523 may be a multipurpose device (e.g., a dual directional coupler) that passes the relatively high amplitude, extremely short duration RF pulse to the TX antenna 410 with minimal insertion loss, while simultaneously providing two low-level outputs to the feedback subsystem 512. One output delivers a forward power signal to the feedback subsystem 512, where the forward power signal is an attenuated version of the RF pulse sent to the TX antenna 410, and the other output delivers a reverse power signal to a different port of the feedback subsystem 512, where reverse power is an attenuated version of the reflected RF energy from the TX Antenna 410.

During the on-cycle time (e.g., while an RF signal is being transmitted to tissue stimulator 102), the RF switch 523 is set to send the forward power signal to feedback subsystem 512. During the off-cycle time (e.g., while an RF signal is not being transmitted to the tissue stimulator 102), the RF switch 523 can change to a receiving mode in which the reflected RF energy and/or RF signals from the tissue stimulator 102 are received to be analyzed in the feedback subsystem 512.

The feedback subsystem 512 of the RF pulse generator module 406 may include reception circuitry to receive and extract telemetry or other feedback signals from tissue stimulator 102 and/or reflected RF energy from the signal sent by TX antenna 410. The feedback subsystem 512 may include an amplifier 526, a filter 524, a demodulator 522, and an A/D converter 520. The feedback subsystem 512 receives the forward power signal and converts this high-frequency AC signal to a DC level that can be sampled and sent to the controller subsystem 514. In this way, the characteristics of the generated RF pulse can be compared to a reference signal within the controller subsystem 514. If a disparity (e.g., an error) exists in any parameter, the controller subsystem 514 can adjust the output to the RF pulse generator 406. The nature of the adjustment can be proportional to the computed error. The controller subsystem 514 can incorporate additional inputs and limits on its adjustment scheme, such as the signal amplitude of the reverse power and any predetermined maximum or minimum values for various pulse parameters.

The reverse power signal can be used to detect fault conditions in the RF-power delivery system. In an ideal condition, when TX antenna 410 has perfectly matched impedance to the tissue that it contacts, the electromagnetic waves generated from the RF pulse generator module 406 pass unimpeded from the TX antenna 410 into the body tissue. However, in real-world applications, a large degree of variability exists in the body types of users, types of clothing worn, and positioning of the antenna 410 relative to the body surface. Since the impedance of the antenna 410 depends on the relative permittivity of the underlying tissue and any intervening materials and on an overall separation distance of the antenna 410 from the skin, there can be an impedance mismatch at the interface of the TX antenna 410 with the body surface in any given application. When such a mismatch occurs, the electromagnetic waves sent from the RF pulse generator module 406 are partially reflected at this interface, and this reflected energy propagates backward through the antenna feed.

The dual directional coupler RF switch 523 may prevent the reflected RF energy propagating back into the amplifier 526, and may attenuate this reflected RF signal and send the attenuated signal as the reverse power signal to the feedback subsystem 512. The feedback subsystem 512 can convert this high-frequency AC signal to a DC level that can be sampled and sent to the controller subsystem 514. The controller subsystem 514 can then calculate the ratio of the amplitude of the reverse power signal to the amplitude of the forward power signal. The ratio of the amplitude of reverse power signal to the amplitude level of forward power may indicate severity of the impedance mismatch.

In order to sense impedance mismatch conditions, the controller subsystem 514 can measure the reflected-power ratio in real time, and according to preset thresholds for this measurement, the controller subsystem 514 can modify the level of RF power generated by the RF pulse generator module 406. For example, for a moderate degree of reflected power the course of action can be for the controller subsystem 514 to increase the amplitude of RF power sent to the TX antenna 410, as would be needed to compensate for slightly non-optimum but acceptable TX antenna coupling to the body. For higher ratios of reflected power, the course of action can be to prevent operation of the RF pulse generator module 406 and set a fault code to indicate that the TX antenna 410 has little or no coupling with the body. This type of reflected power fault condition can also be generated by a poor or broken connection to the TX antenna 410. In either case, it may be desirable to stop RF transmission when the reflected power ratio is above a defined threshold, because internally reflected power can lead to unwanted heating of internal components, and this fault condition means that the system cannot deliver sufficient power to the tissue stimulator 102 and thus cannot deliver therapy to the user.

The controller 542 of the tissue stimulator 102 may transmit informational signals, such as a telemetry signal, through the RX antenna 538 to communicate with the RF pulse generator module 406 during its receive cycle. For example, the telemetry signal from the tissue stimulator 102 may be coupled to the modulated signal on the RX antenna 538, during the on and off state of the transistor circuit to enable or disable a waveform that produces the corresponding RF bursts necessary to transmit to the external (or remotely implanted) pulse generator module 406. The RX antenna 238 may be connected to electrodes 554 in contact with tissue to provide a return path for the transmitted signal. An A/D converter can be used to transfer stored data to a serialized pattern that can be transmitted on the pulse modulated signal from the RX antenna 538 of the tissue stimulator 102.

A telemetry signal from the tissue stimulator 102 may include stimulus parameters, such as the power or the amplitude of the current that is delivered to the tissue from the electrodes 554. The feedback signal can be transmitted to the RF pulse generator module 406 to indicate the strength of the stimulus at the target nerve tissue by means of coupling the signal to the RX antenna 538, which radiates the telemetry signal to the RF pulse generator module 406. The feedback signal can include either or both an analog and digital telemetry pulse modulated carrier signal. Data such as stimulation pulse parameters and measured characteristics of stimulator performance can be stored in an internal memory device within the tissue stimulator 102 and sent on the telemetry signal. The frequency of the carrier signal may be in a range of 300 MHz to 8 GHz.

In the feedback subsystem 512, the telemetry signal can be down modulated using the demodulator 522 and digitized by being processed through the analog to digital (A/D) converter 520. The digital telemetry signal may then be routed to the CPU 530 with embedded code, with the option to reprogram, to translate the signal into a corresponding current measurement in the tissue based on the amplitude of the received signal. The CPU 530 of the controller subsystem 514 can compare the reported stimulus parameters to those held in local memory 528 to verify that the tissue stimulator 102 delivered the specified stimuli to target nerve tissue. For example, if the tissue stimulator 102 reports a lower current than was specified, the power level from the RF pulse generator module 406 can be increased so that the tissue stimulator 102 will have more available power for stimulation. The tissue stimulator 102 can generate telemetry data in real time (e.g., at a rate of 8 kbits per second). All feedback data received from the tissue stimulator 102 can be logged against time and sampled to be stored for retrieval to a remote monitoring system accessible by a health care professional for trending and statistical correlations.

The sequence of remotely programmable RF signals received by the RX antenna 538 may be conditioned into waveforms that are controlled within the tissue stimulator 102 by the control subsystem 542 and routed to the appropriate electrodes 554 that are located in proximity to the target nerve tissue. For instance, the RF signal transmitted from the RF pulse generator module 406 may be received by RX antenna 538 and processed by circuitry, such as waveform conditioning circuitry 540, within the tissue stimulator 102 to be converted into electrical pulses applied to the electrodes 554 through an electrode interface 552. In some implementations, the tissue stimulator 102 includes between two to sixteen electrodes 554.

The waveform conditioning circuitry 540 may include a rectifier 544, which rectifies the signal received by the RX antenna 538. The rectified signal may be fed to the controller 542 for receiving encoded instructions from the RF pulse generator module 406. The rectifier signal may also be fed to a charge balance component 546 that is configured to create one or more electrical pulses such that the one or more electrical pulses result in a substantially zero net charge at the one or more electrodes 554 (that is, the pulses are charge balanced). The charge balanced pulses are passed through the current limiter 548 to the electrode interface 552, which applies the pulses to the electrodes 554 as appropriate.

The current limiter 548 insures the current level of the pulses applied to the electrodes 554 is not above a threshold current level. In some implementations, an amplitude (for example, a current level, a voltage level, or a power level) of the received RF pulse directly determines the amplitude of the stimulus. In this case, it may be particularly beneficial to include current limiter 548 to prevent excessive current or charge being delivered through the electrodes 554, although the current limiter 548 may be used in other implementations where this is not the case. Generally, for a given electrode 554 having several square millimeters of surface area, it is the charge per phase that should be limited for safety (where the charge delivered by a stimulus phase is the integral of the current). But, in some cases, the limit can instead be placed on the current, where the maximum current multiplied by the maximum possible pulse duration is less than or equal to the maximum safe charge. More generally, the current limiter 548 acts as a charge limiter that limits a characteristic (for example, a current or duration) of the electrical pulses so that the charge per phase remains below a threshold level (typically, a safe-charge limit).

In the event the tissue stimulator 102 receives a “strong” pulse of RF power sufficient to generate a stimulus that would exceed the predetermined safe-charge limit, the current limiter 548 can automatically limit or “clip” the stimulus phase to maintain the total charge of the phase within the safety limit. The current limiter 548 may be a passive current limiting component that cuts the signal to the electrodes 554 once the safe current limit (the threshold current level) is reached. Alternatively, or additionally, the current limiter 548 may communicate with the electrode interface 552 to turn off all electrodes 554 to prevent tissue damaging current levels.

A clipping event may trigger a current limiter feedback control mode. The action of clipping may cause the controller to send a threshold power data signal to the RF pulse generator module 406. The feedback subsystem 512 detects the threshold power signal and demodulates the signal into data that is communicated to the controller subsystem 514. The controller subsystem 514 algorithms may act on this current-limiting condition by specifically reducing the RF power generated by the RF pulse generator module 406, or cutting the power completely. In this way, the RF pulse generator module 406 can reduce the RF power delivered to the body if the tissue stimulator 102 reports that it is receiving excess RF power.

The controller 550 may communicate with the electrode interface 552 to control various aspects of the electrode setup and pulses applied to the electrodes 554. The electrode interface 552 may act as a multiplex and control the polarity and switching of each of the electrodes 554. For instance, in some implementations, the tissue stimulator 102 has multiple electrodes 554 in contact with the target neural tissue, and for a given stimulus, the RF pulse generator module 406 can arbitrarily assign one or more electrodes to act as a stimulating electrode, to act as a return electrode, or to be inactive by communication of assignment sent wirelessly with the parameter instructions, which the controller 550 uses to set electrode interface 552 as appropriate. It may be physiologically advantageous to assign, for example, one or two electrodes 554 as stimulating electrodes and to assign all remaining electrodes 554 as return electrodes.

Also, in some implementations, for a given stimulus pulse, the controller 550 may control the electrode interface 552 to divide the current arbitrarily (or according to instructions from the RF pulse generator module 406) among the designated stimulating electrodes. This control over electrode assignment and current control can be advantageous because in practice the electrodes 554 may be spatially distributed along various neural structures, and through strategic selection of the stimulating electrode location and the proportion of current specified for each location, the aggregate current distribution on the target neural tissue can be modified to selectively activate specific neural targets. This strategy of current steering can improve the therapeutic effect for the patient.

In another implementation, the time course of stimuli may be arbitrarily manipulated. A given stimulus waveform may be initiated at a time T_start and terminated at a time T_final, and this time course may be synchronized across all stimulating and return electrodes. Furthermore, the frequency of repetition of this stimulus cycle may be synchronous for all of the electrodes 554. However, the controller 550, on its own or in response to instructions from the RF pulse generator module 406, can control electrode interface 552 to designate one or more subsets of electrodes to deliver stimulus waveforms with non-synchronous start and stop times, and the frequency of repetition of each stimulus cycle can be arbitrarily and independently specified.

For example, a tissue stimulator 102 having eight electrodes 554 may be configured to have a subset of five electrodes, called set A, and a subset of three electrodes, called set B. Set A may be configured to use two of its electrodes as stimulating electrodes, with the remainder being return electrodes. Set B may be configured to have just one stimulating electrode. The controller 550 could then specify that set A deliver a stimulus phase with 3 mA current for a duration of 200 us, followed by a 400 us charge-balancing phase. This stimulus cycle could be specified to repeat at a rate of 60 cycles per second. Then, for set B, the controller 550 could specify a stimulus phase with 1 mA current for duration of 500 us, followed by a 800 us charge-balancing phase. The repetition rate for the set B stimulus cycle can be set independently of set A (e.g., at 25 cycles per second). Or, if the controller 550 was configured to match the repetition rate for set B to that of set A, for such a case the controller 550 can specify the relative start times of the stimulus cycles to be coincident in time or to be arbitrarily offset from one another by some delay interval.

In some implementations, the controller 550 can arbitrarily shape the stimulus waveform amplitude, and may do so in response to instructions from the RF pulse generator module 406. The stimulus phase may be delivered by a constant-current source or a constant-voltage source, and this type of control may generate characteristic waveforms that are static. For example, a constant current source generates a characteristic rectangular pulse in which the current waveform has a very steep rise, a constant amplitude for the duration of the stimulus, and then a very steep return to baseline. Alternatively, or additionally, the controller 550 can increase or decrease the level of current at any time during the stimulus phase and/or during the charge-balancing phase. Thus, in some implementations, the controller 550 can deliver arbitrarily shaped stimulus waveforms such as a triangular pulse, sinusoidal pulse, or Gaussian pulse for example. Similarly, the charge-balancing phase can be arbitrarily amplitude-shaped, and similarly a leading anodic pulse (prior to the stimulus phase) may also be amplitude-shaped.

As described above, the tissue stimulator 102 may include a charge balancing component 546. Generally, for constant current stimulation pulses, pulses should be charge balanced by having the amount of cathodic current should equal the amount of anodic current, which is typically called biphasic stimulation. Charge density is the amount of current times the duration it is applied, and is typically expressed in the units uC/cm². In order to avoid the irreversible electrochemical reactions such as pH change, electrode dissolution as well as tissue destruction, no net charge should appear at the electrode-electrolyte interface, and it is generally acceptable to have a charge density less than 30 uC/cm². Biphasic stimulating current pulses ensure that no net charge appears at the electrode 554 after each stimulation cycle and that the electrochemical processes are balanced to prevent net dc currents. The tissue stimulator 102 may be designed to ensure that the resulting stimulus waveform has a net zero charge. Charge balanced stimuli are thought to have minimal damaging effects on tissue by reducing or eliminating electrochemical reaction products created at the electrode-tissue interface.

A stimulus pulse may have a negative-voltage or current, called the cathodic phase of the waveform. Stimulating electrodes may have both cathodic and anodic phases at different times during the stimulus cycle. An electrode 554 that delivers a negative current with sufficient amplitude to stimulate adjacent neural tissue is called a “stimulating electrode.” During the stimulus phase, the stimulating electrode acts as a current sink. One or more additional electrodes act as a current source and these electrodes are called “return electrodes.” Return electrodes are placed elsewhere in the tissue at some distance from the stimulating electrodes. When a typical negative stimulus phase is delivered to tissue at the stimulating electrode, the return electrode has a positive stimulus phase. During the subsequent charge-balancing phase, the polarities of each electrode are reversed.

In some implementations, the charge balance component 546 uses one or more blocking capacitors placed electrically in series with the stimulating electrodes and body tissue, between the point of stimulus generation within the stimulator circuitry and the point of stimulus delivery to tissue. In this manner, a resistor-capacitor (RC) network may be formed. In a multi-electrode stimulator, one charge-balance capacitors may be used for each electrode, or a centralized capacitors may be used within the stimulator circuitry prior to the point of electrode selection. The RC network can block direct current (DC). However, the RC network can also prevent low-frequency alternating current (AC) from passing to the tissue. The frequency below which the series RC network essentially blocks signals is commonly referred to as the cutoff frequency, and in some embodiments, the design of the stimulator system may ensure that the cutoff frequency is not above the fundamental frequency of the stimulus waveform. In example embodiment 400, the tissue stimulator 102 may have a charge-balance capacitor with a value chosen according to the measured series resistance of the electrodes and the tissue environment in which the stimulator is implanted. By selecting a specific capacitance value, the cutoff frequency of the RC network in this embodiment is at or below the fundamental frequency of the stimulus pulse.

In other implementations, the cutoff frequency may be chosen to be at or above the fundamental frequency of the stimulus, and in this scenario the stimulus waveform created prior to the charge-balance capacitor, called the drive waveform, may be designed to be non-stationary, where the envelope of the drive waveform is varied during the duration of the drive pulse. For example, in one embodiment, the initial amplitude of the drive waveform is set at an initial amplitude Vi, and the amplitude is increased during the duration of the pulse until it reaches a final value k*Vi. By changing the amplitude of the drive waveform over time, the shape of the stimulus waveform passed through the charge-balance capacitor is also modified. The shape of the stimulus waveform may be modified in this fashion to create a physiologically advantageous stimulus.

In some implementations, the tissue stimulator 102 may create a drive-waveform envelope that follows the envelope of the RF pulse received by the RX antenna 538. In this case, the RF pulse generator module 406 can directly control the envelope of the drive waveform within the tissue stimulator 102, and thus no energy storage may be required inside of the tissue stimulator 102, itself. In this implementation, the stimulator circuitry may modify the envelope of the drive waveform or may pass it directly to the charge-balance capacitor and/or electrode-selection stage.

In some implementations, the tissue stimulator 102 may deliver a single-phase drive waveform to the charge balance capacitor or it may deliver multiphase drive waveforms. In the case of a single-phase drive waveform (e.g., a negative-going rectangular pulse), this pulse includes the physiological stimulus phase, and the charge-balance capacitor is polarized (charged) during this phase. After the drive pulse is completed, the charge balancing function is performed solely by the passive discharge of the charge-balance capacitor, where is dissipates its charge through the tissue in an opposite polarity relative to the preceding stimulus. In one implementation, a resistor within the tissue stimulator 102 facilitates the discharge of the charge-balance capacitor. In some implementations, using a passive discharge phase, the capacitor may allow virtually complete discharge prior to the onset of the subsequent stimulus pulse.

In the case of multiphase drive waveforms, the tissue stimulator 102 may perform internal switching to pass negative-going or positive-going pulses (phases) to the charge-balance capacitor. These pulses may be delivered in any sequence and with varying amplitudes and waveform shapes to achieve a desired physiological effect. For example, the stimulus phase may be followed by an actively driven charge-balancing phase, and/or the stimulus phase may be preceded by an opposite phase. Preceding the stimulus with an opposite-polarity phase, for example, can have the advantage of reducing the amplitude of the stimulus phase required to excite tissue.

In some implementations, the amplitude and timing of stimulus and charge-balancing phases is controlled by the amplitude and timing of RF pulses from the RF pulse generator module 406, and in other implementations, this control may be administered internally by circuitry onboard the tissue stimulator 102, such as controller 550. In the case of onboard control, the amplitude and timing may be specified or modified by data commands delivered from the pulse generator module 406.

While the RF pulse generator module 406 and the TX antenna 410 have been described and illustrated as separate components, in some embodiments, the RF pulse generator module 406 and the TX antenna 410 may be physically located in the same housing or other packaging. Furthermore, while the RF pulse generator module 406 and the TX antenna 410 have been described and illustrated as located external to the body, in some embodiments, either or both of the RF pulse generator module 406 and the TX antenna 410 may be designed to be implanted subcutaneously. While the RF pulse generator module 406 and the TX antenna 410 have been described and illustrated as coupled via a wired connection 408, in some embodiments (e.g., where the RF pulse generator module 406 is either located externally or implanted subcutaneously), the RF pulse generator module 406 and the TX antenna 410 may be coupled via a wireless connection.

Other embodiments of positioning devices and tissue stimulation systems are within the scope of the following claims.

What is claimed is:

1. A device for securing a tissue stimulator to a tissue within a body, the device comprising:
a first housing, defining:
a first receptacle configured to grasp the tissue stimulator, and
a protrusion extending away from the receptacle;
a second housing, defining:
a second receptacle configured to grasp the tissue stimulator, and
an opening configured to receive the protrusion to secure the first and second housings together such that the first and second receptacles are aligned to form a channel that surrounds the tissue stimulator and fixes a position of the tissue stimulator relative to the first and second housings; and
an attachment feature by which either or both of the first and second housings can be secured to the tissue with the tissue stimulator carried therein.

2. The device of claim 1, wherein the tissue stimulator is a first tissue stimulator and the channel is a first channel, and wherein the first housing further defines a third receptacle configured to grasp a second tissue stimulator and the second housing further defines a fourth receptacle configured to grasp the second tissue stimulator such the third and fourth receptacles are aligned to form a second channel that surrounds the second tissue stimulator to fix the position of the first tissue stimulator relative to the second tissue stimulator.

3. The device of claim 2, wherein the first and second channels are spaced apart from each other.

4. The device of claim 3, wherein the first and second channels are spaced apart by a distance of about 2 mm to about 15 mm.

5. The device of claim 4, wherein the first and second channels have a length of about 2 mm to about 30 mm.

6. device of claim 1, wherein the attachment feature comprises a through hole by which the device can be sutured to the tissue.

7. The device of claim 1, wherein the attachment feature comprises a textured surface into which the tissue can grow to secure the device to the tissue.

8. The device of claim 1, wherein the attachment feature is defined by one or both of the first and second housings.

9. The device of claim 1, wherein the first and second housings comprise one or more biocompatible polymer materials.

10. The device of claim 1, wherein the first and second housings have a durometer of 40 Shore A to 90 Shore D.

11. The device of claim 1, further comprising a plurality of gripping elements disposed along the first and second receptacles for grasping the tissue stimulator.

12. The device of claim 11, wherein the plurality of gripping elements comprise teeth.

13. The device of claim 11, wherein the plurality of gripping elements have a convex profile.

14. The device of claim 1, wherein the protrusion is configured to clamp the first housing to the second housing.

15. The device of claim 14, wherein the protrusion comprises a lip configured to remain exterior to the opening in the second housing when the first and second housings are attached to each other.

16. A method of using a device to secure a tissue stimulator to a tissue within a body, the method comprising:
grasping the tissue stimulator within a first receptacle of a first housing of the device;
passing a protrusion of the first housing through an opening of a second housing of the device to attach the second housing to the first housing such that a second receptacle of the second housing grasps the tissue stimulator and such that the first and second receptacles align to form a channel that surrounds the tissue stimulator and fixes a position of the tissue stimulator relative to the first and second housings; and
securing either or both of the first and second housings, with the tissue stimulator carried therein, to the tissue at an attachment feature of the device.

17. The method of claim 16, wherein the tissue stimulator is a first tissue stimulator and the channel is a first channel, method further comprising:
grasping a second tissue stimulator within a third receptacle of the first housing of the device;
grasping the second tissue stimulator within a fourth receptacle of the second housing of the device such that the third and fourth receptacles align to form a second channel that surrounds the second tissue stimulator and fixes the position of the first tissue stimulator relative to the second tissue stimulator.

18. The method of claim 16, further comprising positioning the first and second tissue stimulators about 2 mm to about 15 mm from each other.

19. The method of claim 19, further comprising suturing one or both of the first and second housings at a through opening of the device.

20. The method of claim 16, wherein the attachment feature comprises a textured surface into which the tissue can grow to secure the device to the tissue.
